(19) United States
(12) Patent Application Publication
(10) Pub. No.: US 2025/0127438 A1
(43) Pub. Date: Apr. 24, 2025

(54) FIBER COUPLED NETWORK OF SENSORS FOR NEAR-INFRARED SPECTROSCOPY BASED TISSUE OXYGENATION

(71) Applicant: NEC Laboratories America, Inc., Princeton, NJ (US)

(72) Inventors: Sarper OZHARAR, Pennington, NJ (US); Yue TIAN, Princeton, NJ (US); Thomas FERREIRA de LIMA, Princeton, NJ (US); Wataru KOHNO, Princeton, NJ (US); Ting WANG, West Windsor, NJ (US)

(73) Assignee: NEC Laboratories America, Inc., Princeton, NJ (US)

(21) Appl. No.: 18/901,562

(22) Filed: Sep. 30, 2024

Related U.S. Application Data
(60) Provisional application No. 63/543,969, filed on Oct. 13, 2023.

(51) Int. Cl. A61B 5/1455; A61B 5/00

(52) U.S. Cl. CPC A61B 5/14551; A61B 5/002

(57) ABSTRACT

An improved near-infrared spectroscopy based handheld tissue oxygenation scanner and method in which the handheld tissue oxygenation scanner is a small device-like an optical mouse-employing a single transmitter-receiver pair, an optical switch, and a plurality of optical fibers that transmit light into tissue under examination and receive light after interacting with that tissue. The integration of a plurality of optical fibers wherein each individual one provides emission/reception of infrared radiation advantageously provides a higher density of tissue interrogation by the infrared radiation thereby providing faster operation and more information in a given time period. Additional features and advantages of our inventive system and method include computer control and the ability to connect to a variety of computing devices via BlueTooth and other communications techniques.

### CROSS-REFERENCE TO RELATED APPLICATIONS

This application claims the benefit of U.S. Provisional Patent Application Ser. No. 63/543,969 filed Oct. 13, 2023, the entire contents of which is incorporated by reference as if set forth at length herein.

### FIELD OF THE INVENTION

This application relates generally to near-infrared spectroscopy More particularly, it pertains to near-infrared spectroscopy based handheld tissue oxygenation scanner.

### BACKGROUND OF THE INVENTION

As is known in the art, near-infrared spectroscopy (NIRS) is a technology to measure the changes in the oxyhemoglobin and deoxyhemoglobin in both arterial and venous blood, and the total hemoglobin in tissue.

Methods and devices employing NIRS technology send near infrared and infrared (red) light into the tissue and detect any backscattered light. By comparing changes and ratios of intensities of backscattered light, operational methods and devices employing the same may determine the oxy- and deoxy-hemoglobin in the tissue that is illuminated.

Such methods and devices employing NIRS technology are known to be quite useful in diagnostic environments. Accordingly, improvements in such methods and devices would be a welcome addition to the art.

### SUMMARY OF THE INVENTION

An advance in the art is made according to aspects of the present disclosure directed to an improved near-infrared spectroscopy based handheld tissue oxygenation scanner and method.

In sharp contrast to the prior art, our inventive handheld tissue oxygenation scanner is a small device-like an optical mouse-employing a single transmitter-receiver pair, an optical switch, and a plurality of optical fibers that transmit light into tissue under examination and receive light after interacting with that tissue.

Our inventive integration of a plurality of optical fibers wherein each individual one provides emission/reception of infrared radiation advantageously provides a higher density of tissue interrogation by the infrared radiation thereby providing faster operation and more information in a given time period. Additional features and advantages of our inventive system and method include the ability to connect to a variety of computing devices via BlueTooth.

### BRIEF DESCRIPTION OF THE DRAWING

A more complete understanding of the present disclosure may be realized by reference to the accompanying drawing in which:

FIG. 1 is a schematic diagram showing an illustrative side view of a tissue oxygenation scanner according to aspects of the present disclosure; and

FIG. 2 is a schematic diagram showing an illustrative top view of an exemplary optical fiber end point arrangement for a tissue oxygenation scanner according to aspects of the present disclosure.

The illustrative embodiments are described more fully by the Figures and detailed description. The inventions may, however, be embodied in various forms and are not limited to specific embodiments described in the Figures and detailed description.

### DETAILED DESCRIPTION OF THE INVENTION

The following merely illustrates the principles of this disclosure. It will thus be appreciated that those skilled in the art will be able to devise various arrangements which, although not explicitly described or shown herein, embody the principles of the disclosure and are included within its spirit and scope.

Furthermore, all examples and conditional language recited herein are intended to be only for pedagogical purposes to aid the reader in understanding the principles of the disclosure and the concepts contributed by the inventor(s) to furthering the art and are to be construed as being without limitation to such specifically recited examples and conditions.

Moreover, all statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Thus, for example, it will be appreciated by those skilled in the art that any block diagrams herein represent conceptual views of illustrative circuitry embodying the principles of the disclosure.

Thus, for example, it will be appreciated by those skilled in the art that any block diagrams herein represent conceptual views of illustrative circuitry embodying the principles of the disclosure. Similarly, it will be appreciated that any flow charts, flow diagrams, state transition diagrams, pseudo code, and the like represent various processes which may be substantially represented in computer readable medium and so executed by a computer or processor, whether or not such computer or processor is explicitly shown.

The functions of the various elements shown in the Figures, including any functional blocks labeled as “processors”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, network processor, application specific integrated circuit (ASIC), field programmable gate array (FPGA), read-only memory (ROM) for storing software, random access memory (RAM), and non-volatile storage. Other hardware, conventional and/or custom, may also be included.

Software modules, or simply modules which are implied to be software, may be represented herein as any combination of flowchart elements or other elements indicating performance of process steps and/or textual description. Such modules may be executed by hardware that is expressly or implicitly shown.

Unless otherwise explicitly specified herein, the FIGs comprising the drawing are not drawn to scale.

Generally, and as those skilled in the art will understand and appreciate, near-infrared spectroscopy (NIRS) is a powerful analytical technique that uses the near-infrared region of the electromagnetic spectrum (780 nm to 2500 nm) to identify and quantify the chemical composition of a sample substance. It works by measuring how much light at different wavelengths is absorbed by the sample.

NIRS has many advantages over other analytical techniques, such as:
Non-destructive: It does not harm or alter the sample.
Fast: Measurements can be made quickly and easily.
Versatile: It can be used to analyze a wide variety of materials, including liquids, solids, and gases.
Portable: NIR spectrometers can be small and portable, making them suitable for field use.

NIRS has a wide range of applications in various fields, including:
Food and agriculture: Analyzing the quality and composition of food products, monitoring crop growth, and detecting contaminants.
Pharmaceuticals: Identifying and quantifying the active ingredients in drugs, monitoring drug production processes, and ensuring the quality of pharmaceutical products.
Chemical industry: Analyzing the composition of chemicals, monitoring chemical reactions, and optimizing production processes.
Environmental monitoring: Detecting pollutants in air and water, monitoring soil quality, and analyzing the composition of plants.
Medical diagnostics: Measuring blood oxygen levels in the brain, monitoring brain activity, and diagnosing diseases such as cancer.

Overall, NIRS is a powerful and versatile analytical technique that has a wide range of applications. It is a valuable tool for scientists, engineers, and technicians in many different fields.

Advantageously NIRS is a technology that can measure the changes in the oxyhemoglobin and deoxyhemoglobin in both arterial and venous blood, and the total hemoglobin in the tissue.

Devices and methods that employ NIRS send near infrared and red light into a tissue and detect backscattered light. By comparing the changes and ratios of the intensity of backscattered light, the device can determine the oxy- and deoxy-hemoglobin in the tissue that is illuminated.

If such a device is used on the brain tissue, it can penetrate 2-3 cm deep and can be used to track changes in the oxygen consumption in area of the brain that is interrogated by the light signal. In case a large area of the tissue (brain or muscle) needs to be monitored, multiples of such transmitter and receiver pairs need to be used simultaneously to illuminate and monitor this large surface.

However, due to scattering in the biological tissue, independent of the light source (LED or laser), a large tissue area is illuminated, and the detected light cannot be mapped to a single point but instead to a “banana shaped” volume in the tissue. Hence the NIRS systems exhibit relatively low resolution. Additionally, to have a deep penetration of the near-infrared light, the transmitter-receiver pairs should have an average distance of ˜3 cm.

As presently implemented in contemporary applications, each transmitter-receiver pair defines a banana shaped “pixel”. To increase coverage area and to obtain a smoother image, one needs to place more and more transmitter-receiver pairs in a smaller area within the 3 cm distance limitation of the technique. This results in an increased number of transmitters and detectors, hence the complexity of the system and the cost. Also, the device will get bulkier and heavier, which may prevent its usefulness in mobility. Consequently, increasing the resolution of NIRS tissue scanning systems and methods remains difficult and unfulfilled.

As noted previously, an aspect of the present disclosure is directed to an improved near-infrared spectroscopy-based tissue oxygenation scanner and method that employs an optical switch and a plurality of optical fibers for near-infrared spectroscopy tissue interrogation. Operational coordination among/between the elements is performed by a processor.

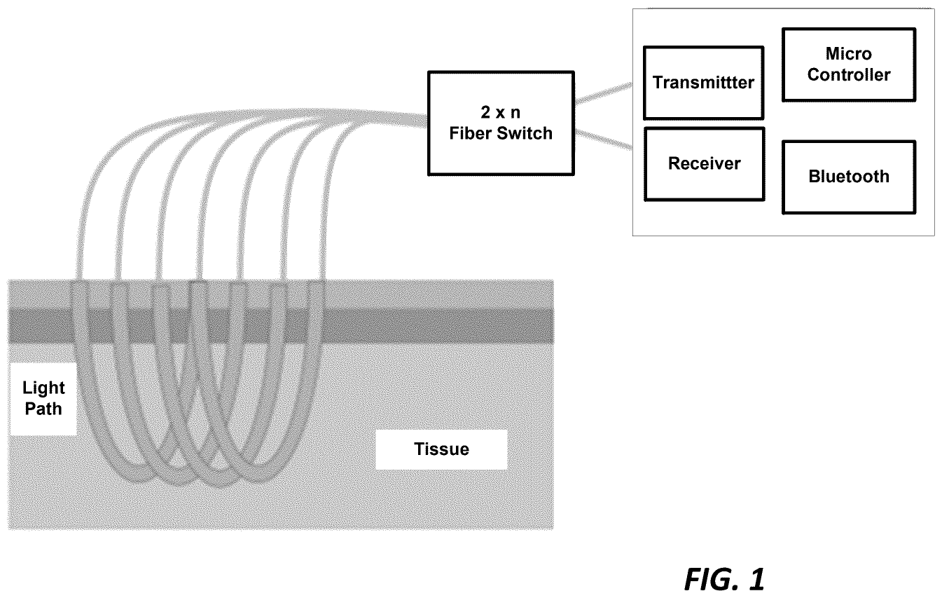

FIG. 1 is a schematic diagram showing an illustrative side view of a tissue oxygenation scanner according to aspects of the present disclosure.

As shown in that figure, a near-infrared tissue scanner according to the present disclosure includes a detector includes a single transmitter/receiver pair, a plurality of optical fibers that deliver interrogation near-infrared light into tissue under examination and receive light scattered from that examined tissue, and an optical fiber switch interposed in an optical path and connecting the transmitter/receiver pair to the plurality of optical fibers that deliver/receive the near-infrared light/scattered light to/from the tissue.

As those skilled in the art will understand and appreciate, our inventive scanner and method uses multiple optical fibers to transmit the light into the tissue and to collect light scattered from the tissue. Advantageously, with our inventive scanner as illustratively shown in FIG. 1, the multiple optical fibers are optically connected to a single transceiver (transmitter/receiver pair) by a fiber switch. Consequently, our inventive scanner exhibits a simplified design as compared with contemporary scanners.

Operationally, at a given time, the transmitter is optically connected to one of the plurality of optical fibers and the receiver is connected to another one of the plurality of optical fiber through the action of the optical switch which, in the figure, is illustratively shown as a 2×n fiber switch.

The two optical fibers—the one that is optically connected to the transmitter and the one that is optically connected to the receiver are located approximately 3 cm away from one another at the surface of the tissue. That is to say, the two optical fibers constitute a transmitter-receiver pair.

We note further that an end of the optical fiber optically connected to the transmitter is located at an illumination point of the tissue under examination and substantially 3 cm away an end of the other optical fiber of the transmitter-receiver pair is located to detect/receive light that was emitted from the transmitter and scattered by the tissue.

The scattered light is conveyed by the optical fiber to the receiver via the optical switch, and scattering data is derived. Once the scattering data is acquired, the transmitter and the receiver of the scanner are optically connected to another fiber pair, and the process continues until a complete scan of the entire tissue under investigation is performed.

As noted and shown in the FIG. 1, our inventive NIRS scanner employs only one transmitter (LED or laser at multiple wavelengths) and one receiver. These are connected to a 2×n fiber optic switch which directs the light emitted from the transmitter to one of the fibers and illuminates the tissue under examination.

Simultaneously, the switch operates to couple the receiver to another fiber having an end at ˜3 cm away (or any other distance if needed) from the light emission end of the transmitter coupled fiber. In a preferred embodiment the transmitter is a multi-wavelength source. Since the transmitter is a multi-wavelength source, our inventive optical fiber configuration permits efficient spectral measurements and information about an illuminated part of the tissue.

Subsequently, the optical fiber switch is configured to another optical fiber pair and repeats the process. As a result, the tissue is scanned finely in 2-Dimensions, for which the scanning is limited by the number and placement of optical fibers interrogating the tissue

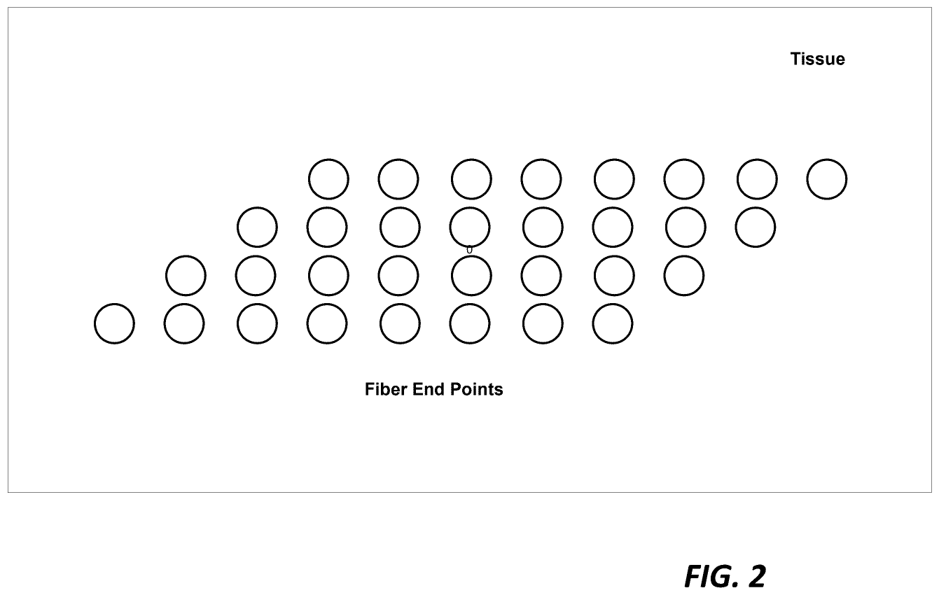

FIG. 2 is a schematic diagram showing an illustrative top view of an illustrative optical fiber end point arrangement for a tissue oxygenation scanner according to aspects of the present disclosure.

With reference to that figure, one may readily recognize some particularly distinguishing aspects of the improved near-infrared spectroscopy based tissue oxygenation scanner according to the present disclosure.

In particular, note that as shown in FIG. 2, each end of any one of the plurality of optical fibers may serve as both a transmitter and a receiver if needed. Additionally, this aspect and the high density of the optical fibers employed guarantee that a transmitter fiber to receiver fiber distance is less than the ˜3 cm distance, thereby producing much higher resolution and precision measurements than possible with conventional scanners.

An illustrative computer architecture of a tissue oxygenation scanner according to aspects of the present disclosure may be integrated into a variety of sensor probe configurations, and may be implemented via discrete circuitry elements or one or more integrated circuitry components. The computer system may comprise, for example, a computer running any of a number of operating/control programs. The above-described methods of the present disclosure may be implemented on the computer system as stored program control instructions.

Such an illustrative computer system includes a processor, memory, storage device, and input/output structure. One or more input/output devices may include a display. One or more busses typically interconnect the components. The processor may be a single or multi core. Additionally, the system may include accelerators etc further comprising the system on a chip.

The processor executes instructions in which embodiments of the present disclosure may comprise steps described in one or more of the Drawing figures. Such instructions may be stored in memory or storage device. Data and/or information may be received and output using one or more input/output devices.

The memory may store data and may be a computer-readable medium, such as volatile or non-volatile memory. The storage device may provide storage for the system including for example, the previously described methods. In various aspects, storage device may be a flash memory device, a disk drive, an optical disk device, or a tape device employing magnetic, optical, or other recording technologies.

Input/output structures may provide input/output operations for the system and include any number of structures previously described including wireless circuitry, light source(s), detector circuitry, optical tracker circuitry, and inertial (IMU) circuitry.

At this point, while we have presented this disclosure using some specific examples, those skilled in the art will recognize that our teachings are not so limited. Accordingly, this disclosure should only be limited by the scope of the claims attached hereto.

1. A fiber coupled tissue oxygen scanner comprising
an optical transceiver including a transmitter and receiver;
a 2×n optical switch in optical communication with the optical transceiver; and
a plurality of optical fibers in optical communication with the 2×n optical switch;
wherein the fiber coupled tissue oxygen scanner is configured such that the one transmitter and the one receiver are optically connected to multiple optical fibers of the plurality of optical fibers; and
wherein at a given time the transmitter is optically connected to one of the optical fibers of the plurality of optical fibers and the receiver is optically connected to another one of the optical fibers of the plurality of optical fibers, the one optical fiber of the plurality of optical fibers connected to the transmitter and the one optical fiber of the plurality of optical fibers connected to the receiver constituting a transmitter-receiver pair;
wherein light generated by the transmitter is emitted from one of the optical fibers of the transmitter-receiver pair, and received by the other one of the optical fibers of the transmitter-receiver pair after it is introduced into animal tissue and scattered by that tissue.

2. The fiber coupled tissue oxygen sensor of claim 1 wherein the light generated by the transmitter is near-infrared light.

3. The fiber coupled tissue oxygen sensor of claim 2 wherein the two optical fibers of the transmitter-receiver pair are separated by a distance of less than 3 cm.

4. The fiber coupled tissue oxygen sensor of claim 3 wherein the scattered near infrared light is indicative of oxy- and deoxy-hemoglobin in the tissue.

5. The fiber coupled tissue oxygen sensor of claim 4 further comprising a controller configured to sequence light transmission and reception to a plurality of transmitter-receiver pairs of optical fibers, one pair at a time.

6. The fiber coupled tissue oxygen sensor of claim 5 further comprising communications circuitry that communicates the oxy- and deoxy-hemoglobin indications to another system.

7. The fiber coupled tissue oxygen sensor of claim 6 wherein the communications circuitry communicates the oxy- and deoxy-hemoglobin indications wirelessly.

8. The fiber coupled tissue oxygen sensor of claim 7 wherein the wireless communications include Bluetooth communications.

9. The fiber coupled tissue oxygen sensor of claim 8 wherein the 2×n optical switch is an m×n optical switch and includes m/2 transmitters and m/2 receivers.

10. The fiber coupled tissue oxygen sensor of claim 6 configured to scan all the transmitter-receiver pairs of optical fibers without physically moving the optical fibers.
